(19) United States
(12) Patent Application Publication
(10) Pub. No.: US 2009/0319007 A1
(43) Pub. Date: Dec. 24, 2009

(54) Shocking device having a time-based monitoring and recording circuit

(76) Inventor: James F. McNulty, JR., Las Vegas, NV (US)

Correspondence Address: LEONARD TACHNER, A PROFESSIONAL LAW;CORPORATION, 17961 SKY PARK CIRCLE, SUITE 38-E, IRVINE, CA, 92614, US

(21) Appl. No.: 12/214,653

(22) Filed: Jun. 20, 2008

(51) Int. Cl. A61N 1/08; F41B 15/04

(52) U.S. Cl. 607/63; 361/232

(57) ABSTRACT

An electronic shocking device comprising a voltage drop detector which signals an integrated circuit to record the occurrence of a shock when the device voltage drops within a preset tolerance corresponding to a recommended level of shocking current. The voltage drop detector or integrated circuit may also activate a display to signal the device operator that the device is actually shocking a subject.

### BACKGROUND OF THE INVENTION

1. Field of the Invention

The invention relates generally to the field of shocking devices containing circuitry intended to record and signal an actual shocking event.

2. Prior Art

Numerous types of devices designed to shock humans and other animals exist. The devices range from medical devices, requiring pre-market notification to or pre-market approval from the United States Food and Drug Administration, to weapons, intended to subdue and capture. Descriptions of some of the many general classes of shocking therapeutic medical devices may be found at 21 CFR Parts 876 (Gastroenterology and Urology Devices), 884 (Obstetrical & Gynecological Devices) and 890 (Physical Medicine Devices). Many United States patents for shocking weapons for subduing and capturing human and other animal targets have issued. These patents include U.S. Pat. No. 3,803,463 to Cover, U.S. Pat. No. 4,688,140 to Hammes and U.S. Pat. No. 5,841,622 to McNulty. Such weapons or restraints are manufactured and sold by Taser International, Inc. of Scottsdale, Ariz. such as the model X26 Taser™, by Stinger Systems, Inc. of Tampa, Fla. such as the ICE™ shield and by Bestex Company, Inc. of Los Angeles, Calif. such as the Dual Defense System™ remote contact stun gun and the Space Thunder™ stun gun.

As manufactured and sold, many of the shocking devices comprise digital displays or other circuitry designed to indicate and/or record the devices' therapeutic or disabling operation and/or the devices' time of therapeutic or disabling operation and/or to discontinue the devices' operation after a time of operation. Uniformly, however, these designs do not achieve their intended purpose, as the designs indicate/record a device's energization and not necessarily the time of its shock, the intensity of its shock or its shock. The digital displays and other circuitry indicate and record even when one or more of the devices electrodes is not in electrical contact or in secure electrical contact with the subject intended to be shocked by the device.

Particularly illustrative of this problem is U.S. Pat. No. 7,234,262 to Smith for apparatus for recording operation of a Taser weapon. The '262 inventions are embodied in Taser International, Inc.'s M26 and X26 model Tasers. It appears from the claims and specification of the '262 patent that the object of the invention is to insure that the weapon shocks for a duration adequate to disable its target. See '262 patent Claim 1 “microprocessor programmed to (1) track date and time . . . (3) to maintain for a period the current from the power supply, and (4) to record tracked date and time . . . ”.

As claimed in each independent claim of the '262 patent, however, the invention will record on each occasion that the Taser's shocking circuit is energized. This does not necessarily provide a record that a target subject was actually shocked during the weapon's time of energization. The high-tension Taser currents can complete alternate circuit paths through atmosphere without shocking, once the weapons are activated if the target path has a higher impedance. See '262 patent at Column 5, lines 8-29 describing high voltage arc detonation of cartridge propellant. See also U.S. Pat. No. 5,654,867 to Murray at Column 6, lines 36-49, where an apparatus uses an alternative atmospheric discharge path to signal a failure to complete a shocking circuit. The '262 patent describes no apparatus or method for initiating recording only when the Taser device is actually shocking.

Monitoring circuits can also be employed as safety circuits for shocking devices. IEC (International Electrical Commission, Rue Devarembe, P.O. Box 131, CH-1211, Geneva 20, Switzerland) and other safety standards or thresholds for shocks are calculated upon time based electrical safety equations, which are only valid for seconds. The time base compensates for shock induced homeostatic imbalances, which rapidly diminish particular organs and organ systems ability to endure the shocks. Recording of the actual shocks is, therefore, desirable for continued treatment and serves a variety of other social purposes.

### SUMMARY OF THE INVENTION

The present invention comprises an electronic shocking device, comprising a voltage drop detector which signals an integrated circuit to record the occurrence of a shock when the device voltage drops within a preset tolerance corresponding to a prescribed or recommended level of shocking current. The voltage drop detector or integrated circuit can also activate a display to signal the device operator that the device is shocking a target subject. Any number of circuit configurations known in the art can readily be assembled by one skilled in circuit design to construct the voltage detector. For example, a simple circuit comprising a voltage divider and comparator could signal a voltage drop. Any number of integrated circuits known in the art from circuits comprising counters to microprocessor calendar clocks can easily be assembled by one skilled in the art of digital circuit design into a circuit for recording the occurrence of a signal generated by the voltage drop detector.

It is therefore a principal object of the invention to provide an apparatus that can accurately indicate the occurrence of a shock from a shocking device, so that the device operator can determine whether or not the shock is occurring at the prescribed or recommended current intensity and occurring for the prescribed or recommended duration.

It is another object of the invention to provide an apparatus that overcomes the inaccuracy inherent with manual recording by automatically recording the occurrence of a shock from a shocking device to provide medical or other records.

It is yet another object of the present invention to provide a safety feature which relies on current-induced voltage reduction to terminate shocking current in the event of an unsafe level of such current.

It is still another object of the invention to provide a circuit for a shocking device which enables calculation of the internal electrical resistance of a target and changes thereof.

### BRIEF DESCRIPTION OF THE DRAWINGS

The aforementioned objects and advantages of the present invention, as well as additional objects and advantages thereof, will be more fully understood herein after as a result of a detailed description of a preferred embodiment when taken in conjunction with the following drawings in which:

FIG. 1 is a block schematic of the invention configured as a shocking weapon, intended to subdue and capture;

FIG. 2A is an operational flow chart for the invention, operated as a weapon to subdue and capture. FIG. 2B is an operational flow chart for the invention, operated as a restraint or medical device; and

FIG. 3 is a schematic block diagram of a voltage drop detector which may be used in the present invention.

### DETAILED DESCRIPTION OF PREFERRED EMBODIMENTS

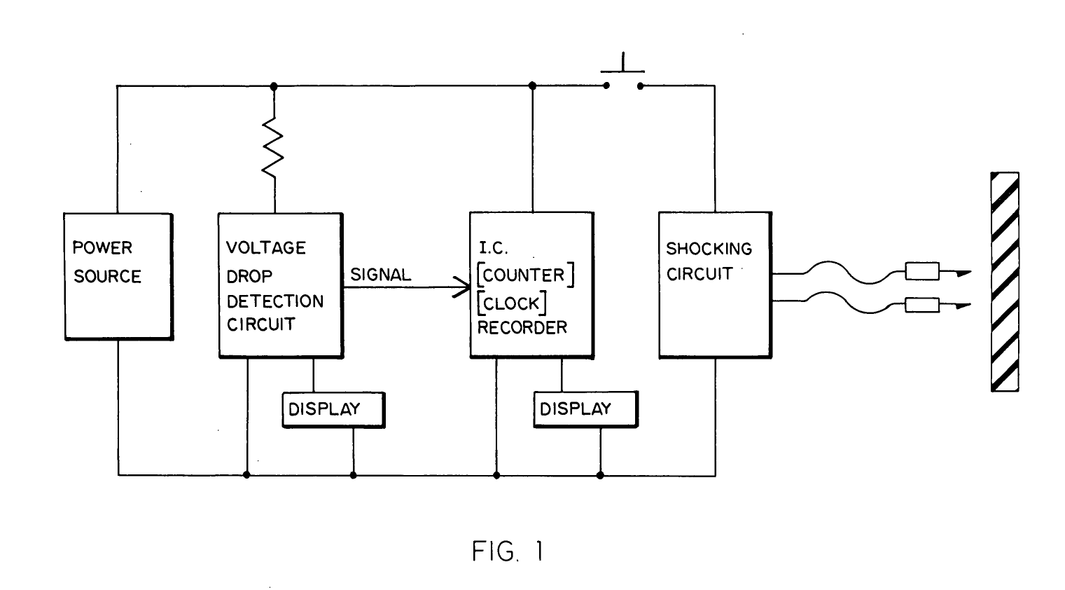

Referring to the accompanying drawings, and to FIG. 1 in particular, it will be seen that the general concept of the invention is disclosed therein. More specifically, a voltage drop detection circuit and a counter or clock-based I.C. recorder, each connected to a display, are interposed between a power source and a high voltage shocking circuit. In a weapon-based configuration, the shocking circuit is adapted for propelling a pair of wire-tethered electrode darts to a remote target. If both such electrode darts successfully impact and adhere to the remote target, the high voltage (i.e., 50 K VOLTS) generated at the shocking circuit, causes an electric current to flow through the target via the wire-tethered darts to disable the target.

The inventive concept hereof is based upon such current flow causing a voltage drop across the target between the two darts. Those having knowledge in the art of electric circuits will appreciate that if, for any reason, current does not flow through the target (such as when one or both darts do not make proper contact with the target) there will be no voltage drop between the darts. Thus, a voltage drop across the darts is very likely to be indicative of an actual shocking of the target rather than merely generation of a high voltage at the shocking circuit. A high voltage at the output of the shocking circuit would occur whenever the power source of FIG. 1 is connected electrically to the shocking circuit, even without current flow through the darts and the target.

A voltage drop at the output of the shocking circuit is reflected as a commensurate voltage drop at the input to the shocking circuit. Therefore, as shown in FIG. 1, a voltage drop detection circuit may be connected at the shocking circuit input to detect a voltage drop as an indication of an actual shocking event. The occurrence of a shocking event may be signaled to a display to alert a user and may be signaled to a counter or clock to record either a count or a time of day when the shocking event commenced. That count or time may also be sent to the display to provide a visual indication of the count or time when the shocking event began.

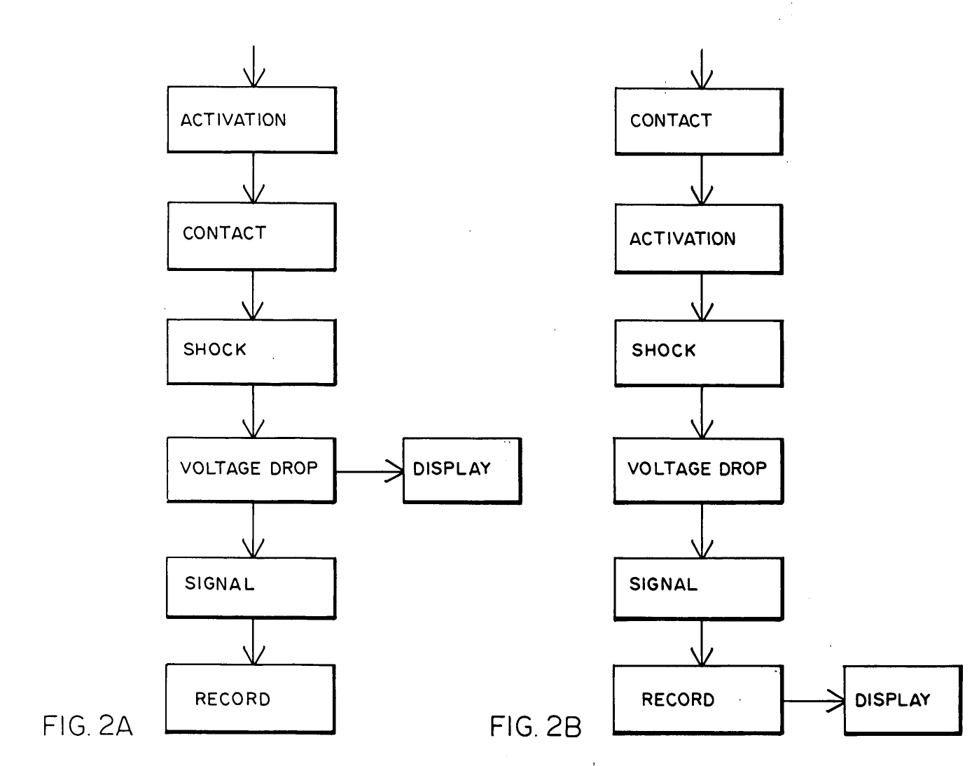

FIGS. 2A and 2B illustrate sequences of events respectively, for weapon and stimulator or restraint versions of an electrical discharge apparatus in which the present invention is employed. FIG. 2A illustrates that for a weapon version, the first event is activation of the apparatus which applies a high voltage to the wire-tethered darts and propels them toward a remote (i.e., 10 to 20 feet) target to impart a disabling electrical discharge into the target. If the darts both successfully impact the target, the corresponding events are contact and shock as depicted in FIG. 2A. As previously described, if an actual shock is occurring at this point, current flows through the wire-tethered darts and through the target, thereby causing a voltage drop which the present invention senses through the voltage drop detector and then displays that event. Simultaneously, a signal is sent to instigate recording of a count or time of the voltage drop event as noted in FIG. 2A.

FIG. 2B illustrates that for a stimulator or restraint version of the invention, the first event is contact with the person to be stimulated or restrained and then there is activation after contact and then shock following activation. Voltage drop detection occurs upon initiation of shock and such detection results in a signal to enable recording of count or time which may be displayed. It will be understood that in a restraint scenario, there may be a long period of time (i.e., hours or days) between contact, activation and shock, depending upon the configuration and purpose of the restraining device.

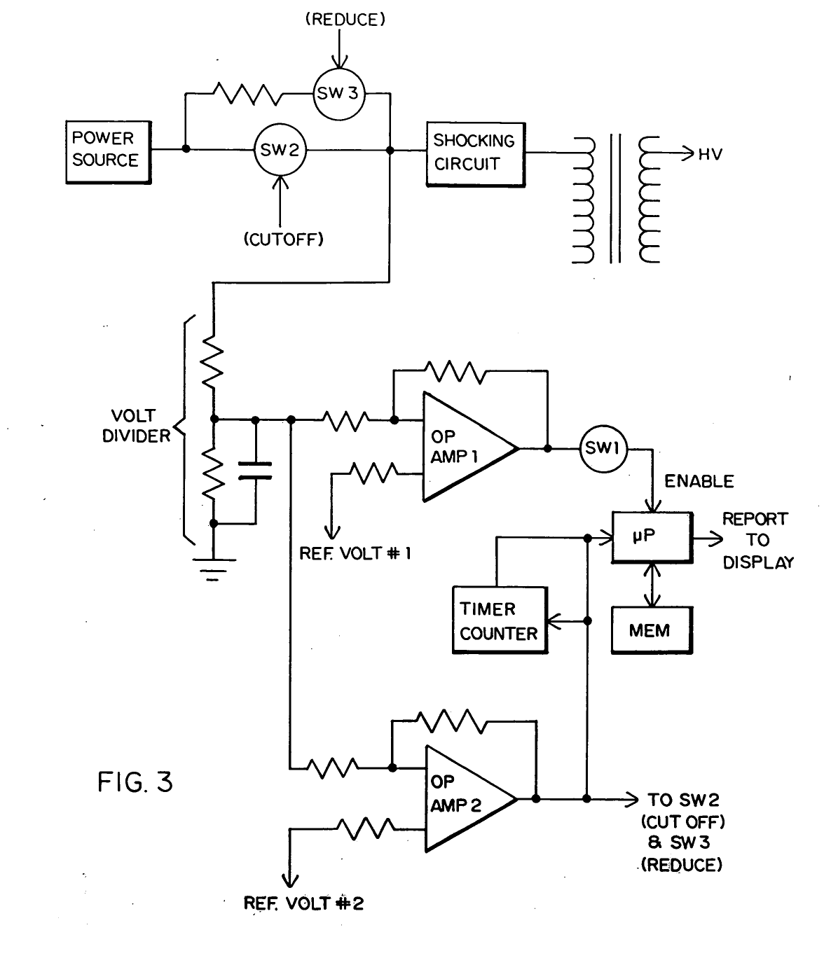

An example of a voltage drop detector is illustrated in FIG. 3. As seen therein, the input to a high voltage transformer is applied to a voltage divider having a smoothing capacitor. The divider sub-voltage is applied as one input to an operational amplifier OPAMP1, the second input to which is a first reference voltage REFVOLT#1. This first reference voltage is selected to correspond to the divided transformer input voltage reduced by a current output corresponding to a shocked target event. Only after the divider voltage drops below the first reference voltage does the output of OPAMP1 produce the appropriate magnitude and polarity signal to close switch SW1 to enable microprocessor μP to transfer a count or time to memory chip MEM and issue a report of the count or time to a display. OPAMP1 operates as a comparator.

FIG. 3 also shows an optional safety addition wherein a second reference voltage REFVOLT#2 is applied to a second operational amplifier OPAMP2. The second reference voltage may be selected to correspond to a higher voltage drop indicating a greater current flow through the target wherein continued flow could cause injury to the target. The output of OPAMP2 can be used to open a cutoff switch SW2 to cease operation of the device. It may also be sent to the timer/counter and to the microprocessor μP to record the count or time of such a cutoff. OPAMP2 also operates as a comparator. The output of OPAMP2 may also be used to open SW2 while closing SW3 to only reduce the shocking rather than to terminate the shocking.

It will now be apparent that the present invention meets the aforementioned object by providing a device for use in shocking products for monitoring and recording when a shocking event actually takes place. An optional feature thereof terminates shocking if the shocking current exceeds a preset safe magnitude. The scope hereof is to be limited only by the appended claims.

I claim:

1. A device for shocking humans and other animals comprising a circuit for signaling the recording of the occurrence of the shock as a time when the shock voltage drops to a level corresponding to a prescribed or recommended value of shocking current.

2. A device for shocking humans and other animals comprising a circuit for signaling and displaying a time when a voltage for shocking drops to within a tolerance corresponding to a prescribed or recommended value of shocking current.

3. The device for shocking recited in claim 2 further comprising a safety circuit for terminating said shocking when said shock voltage drops to a magnitude indicating an unsafe value of shocking current.

4. The device for shocking recited in claim 2 further comprising a safety circuit for reducing the intensity of said shocking when said shock voltage drops to a magnitude indicating an unsafe value of shocking current.

5. In an electrical discharge shocking device configured with a high voltage transformer and a power source for disabling remote targets by propelling a pair of wire-tethered contact darts to impact the target by causing an electric current to flow between the darts and through the target, a circuit for sensing such current flow to record commencement of actual shocking of the target; the circuit comprising:
a voltage drop detector connected to said power source for detecting a voltage reduction caused by said current flow;
a timer having a running clock;
a memory device connected for receiving and storing a signal representative of said clock time;
said detector, said counter and said memory device being connected for generating a record of a time of the event of a voltage reduction.

6. The circuit recited in claim 5 wherein said voltage drop detector is configured for causing a record of said time only when said voltage reduction is at a selected threshold.

7. The circuit of claim 5 further comprising a display for receiving said signal representative of said time and visually displaying said time.

8. The circuit of claim 4 further comprising a voltage divider connected to an output of said power source and a comparator connected to receive a reference signal corresponding to a detected voltage drop indicative of a shocking event of a target and to receive a signal from said voltage divider for comparison with said reference signal.
